(19) United States
(12) Patent Application Publication
(10) Pub. No.: US 2004/0062764 A1
(43) Pub. Date: Apr. 1, 2004

(54) Chemoprotectant for gastric toxicity

(76) Inventors: Edward A. Neuwelt, Portland, OR (US); Leslie Muldoon, Tigard, OR (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/363,150

(22) PCT Filed: Aug. 30, 2001
(86) PCT No.: PCT/US01/27296

(51) Int. Cl. A61K 39/395; A61K 31/7048; A61K 31/704; A61K 31/56; A61K 31/4745; A61K 31/337

(52) U.S. Cl. 424/132.1; 514/562; 424/649; 424/623; 514/34; 514/27; 514/283; 514/449; 514/171

(57) ABSTRACT

There is disclosed a method and pharmaceutical composition for treating or mitigating the side effects of cytotoxic cancer therapy for carcinoma-type cancers tumors including administering a thiol-based chemoproctectant agent and administering a cytotoxic agent having a targeting means to the Lewis Y glycoproteins.

The present invention was partially supported under NM grant #NS33618. The United States Government may have certain rights in this invention.

### TECHNICAL FIELD OF THE INVENTION

The invention provides chemoprotectant agents that may be administered in conjunction with cytotoxic agents that target carcinoma type cancers. More particularly, the cytotoxic agent targets Lewis Y glycoproteins on gastric epithelium, with or without addition of conventional chemotherapeutic agents. Intra-arterial administration of a thiol-based chemoprotective agent will target the cytotoxic agent to reduce immunoconjugate and chemotherapy side effects without decreasing anti-tumor efficacy.

### BACKGROUND OF THE INVENTION

N-acetylcysteine (NAC) is an analog of cysteine with strong anti-oxidant and free radical scavenging activity. In addition to direct chemoprotective activity, when administered to a mammal NAC is deacylated and enters a cellular synthetic pathway for production of glutathione. Glutathione is involved in many cellular processes that may have importance for the resistance of tumors to cytotoxic drugs, including anti-oxidant, drug conjugation, and drug extrusion. Thus, NAC can mimic short term effects of glutathione as well as increasing glutathione for later protective activity. This is especially important when intracellular glutathione is artificially reduced in an effort to enhance the cytotoxic properties of chemotherapeutic drugs, by pretreatment with buthionine sulfoximine (BSO).

A potential problem with any chemoprotectant is the possibility of deactivating the anti-tumor effect of the chemotherapy or radiation therapy. The goal of chemoprotection is to reduce unwanted toxicities of chemotherapy or radiotherapy without affecting efficacy. Therefore, there is a need in the art to improve pharmacokinetics and biodistribution of chemoprotectant agents so that they will be more effective if they can be delivered in a tissue-specific manner. In other words to maximize their deliver, to the breast, gastrointestinal tract, lung, cervix, and ovary while minimizing systemic delivery.

There are several thiol-based chemoprotectant agents that contain a thio, thiol, aminothiol or thioester moiety. These include N-acetyl cysteine (NAC), sodium thiosulfate (STS), GSH ethyl ester, D-methionine, and Ethyol (WR7921). Ethyol is also marketed in the United States under the generic name of Amifostine. GSH ethyl ester is an experimental thiol not yet marketed for clinical use, but is representative of the class of thiols that is converted directly to glutathione.

NAC is currently marketed in the United States under an orphan indication for oral and intra venous (iv) administration for overdosing with acetaminophen. NAC has also been shown to be a chemoprotectant when administered in combination with a vanadate compound (U.S. Pat. No. 5,843,481; and Garbo (ed) Semin. Oncol. 10 [Suppl 1]56-61, 1983). In addition, NAC has been shown to be a mucoregulatory drug used for the treatment of chronic bronchitis (Grassi and Morandini, Eur. J. Clin. Pharmacol. 9:393-396, 1976; Multicenter Study Group, Eur. J. Respir. Dis. 61: [Suppl.]93-108, 1980; and Borman et al., Eur. J. Respir. Dis. 64:405-415, 1983).

In plasma, NAC can be present in its intact, reduced forms as well as in various oxidized forms. It can be oxidized to a disulfide by reacting with other low molecular weight thiols, such as cysteine and glutathione. NAC can be oxidized by reaction the thiol groups of plasma proteins. There are bioanalytical methods for the determination of NAC in plasma, including Cotgreave and Moldeus, Biopharm. Drug Disp. 8:365-375, 1987; and Johansson and Westerlund, J. Chromatogr. 385:343-356, 1986 that also permit a determination of other forms of NAC. Moreover, cysteine and cystine have been identified as major metabolites of NAC. The excreted urinary product was inorganic sulfate together with small amounts of taurine and unchanged NAC. According to the label indications for NAC manufactured by American Regent Laboratories Shirley, N.Y.), vials of NAC are produced as a sterile solution for oral administration diluted with water or soft drinks. NAC is initially diluted in the venous pool when administered iv and then rapidly eliminated from the systemic circulation by the liver. Thus, very little of the initial dose of NAC is available to systemic tissues for entry into the glutathione pathway and potential chemoprotection.

Another thiol-containing chemoprotectant is sodium thiosulfate or STS. Its chemical formula is Na₂S₂O₃ and it has been used clinically for cyanide poisoning and for nephrotoxicity caused by cisplatin. STS is cleared rapidly from circulation primarily by the kidney. The plasma half life after a bolus injection is about 17 minutes. STS can also inactivate platinum agents due to a covalent binding to platinum agents at molar excess >40:1 (STS:platinum). STS is currently used as a chemoprotectant against carboplatin chemotherapy-induced hearing loss (Neuwelt, JPET 1998).

Tumor selective monoclonal antibodies (mAbs) can be used as delivery systems for chemotherapeutic agents, toxins, and enzyme prodrug therapies based on their potential to discriminate neoplastic cell populations relative to normal tissues. A murine mAb, BR96 (IgG1) has been developed which binds to a Lewis Y (Le^{y})-related antigen abundantly expressed at the surface of cells from carcinomas of the lung, breast, ovary and colon while having low reactivity with most normal human tissues (Trail et al. Cancer Res. 59:5693-5700, 1992; Trail et al., Science 261:919-915, 1993. Remsen et al., Neurosurgery, 46:704-709, 2000). The BR96 antibody was conjugated to doxorubicin (DOX) to produce a targeted immunoconjugate. DOX is a broad spectrum antitumor agent frequently used in the treatment of leukemia, breast carcinoma and other cancers, but its efficacy is limited by dose dependent toxicities including bone marrow suppression and cardiotoxicity. The conjugation of the drug to the antibody produced an immunoconjugate, BR96-DOX, with reduced systemic toxicity, and with high specificity against carcinomas that express the Lewis Y antigen. BR96-DOX has been shown to be an effective and safe agent against several tumor types growing as subcutaneous transplants in animal models including human lung adenocarcinoma, colon carcinoma, and breast carcinoma. BR96-DOX, in combination with conventional chemotherapeutic agents such as carboplatin or Taxol (pacletaxel), has synergistic antitumor effect. Next generation antibodies targeting the Lewis Y antigen should also be effective immunoconjugates.

Unfortunately, normal human gastric cells can express the Lewis Y antigen. Therefore, the dose-limiting toxicity of BR96-DOX is gastro-intestinal toxicity or gastritis (Seleh et al., J. Clin. Oncol. 2000). Similar gastritis can be expected from any immunoconjugate that targets the Lewis Y antigen. This GI toxicity must be reduced for this effective experimental approach to be successful in clinical trials.

Immunoconjugate toxicity may be increased by combination with conventional chemotherapy. However, conventional chemotherapy does not induce gastritis on its own. NAC protects against chemotherapy induced systemic toxicity, not inclusive of gastric toxicity.

Therefore, there is a need in the art to find better ways to use thiol-based chemoprotectants, such as NAC and STS, and to take advantage of their pharmacokinetic properties. There is a need in the art to reduce BR96-DOX toxicity in patients' gastric cells. There is a need to reduce the GI toxicity of all Lewis Y targeting immunoconjugates, with or without addition of other commonly used chemotherapeutic agents. There is also a need to reduce end-organ toxicity so that higher dose chemotherapeutic treatment regimens can be used against head and neck as well as brain tumors with limited drug access, that avoid dose-limiting side effects.

### SUMMARY OF THE INVENTION

A method for treating or mitigating the side effects of a cytotoxic cancer therapy for carcinoma type cancers is described. One or a plurality of cytotoxic agents and a thiol-based chemoprotectant agent are administered intra-arterially, wherein the intra-arterial administration is through a catheter placed into an artery that provides blood flow to an organ most susceptible to toxic side effects of the cytotoxic agent. In one embodiment, the thiol-based chemoprotectant agent is a compound selected from the group consisting of N-acetyl cysteine (NAC), sodium thiosulfate (STS), GSH ethyl ester, D-methionine, Ethyol, and combinations thereof. In another embodiment, the cytotoxic agent is selected from the group consisting of chimeric anti-Lewis Y monoclonal antibodies conjugated to a cytotoxic agent, used either alone or in combination with unconjugated, platinum compounds, taxanes (e.g., paclitaxel), steroid derivatives, anti-metabolites, vinca alkaloids, adriamycin and doxarubicin, etoposide, arsenic derivatives, intercalating agents, alkylating agents (e.g., melphalan) and combinations thereof. Preferably, the cytotoxic agent is a monoclonal antibody to the Lewis Y glycoprotein. In a preferred embodiment, the monoclonal antibody is BR96-Doxorubicin. Most preferably, the dose of the thiol-based chemoprotectant agent per procedure is from about 200 mg/m² to about 40 g/m². Most preferably, the daily dose of NAC agent during chemotherapy is from about 400 mg/m² to about 1200 mg/m².

A pharmaceutical composition for treatment of carcinoma type cancers for administration via arterial catheter including a first agent that is a cancer cytotoxic agent and a second agent administered intra-arterially is disclosed, wherein the first agent is a cytotoxic compound that is used for cancer chemotherapy but is dose-limited due to side effects, and the second agent is a thiol-based chemoprotectant agent. In one embodiment, the first agent is selected from the group consisting of chimeric anti-Lewis Y monoclonal antibodies conjugated to a cytotoxic agent used either alone or in combination with unconjugated, platinum compounds, taxanes (e.g., paclitaxel), steroid derivatives, antimetabolites, vinca alkaloids, adriamycin and doxarubicin, etoposide, arsenic derivatives, intercalating agents, alkylating agents (such as melphalan), and combinations thereof. In a preferred embodiment, the chimeric monoclonal antibody is BR96-Doxorubicin. Preferably, the second agent is administered in a pyrogen-free sterile solution. Preferably, the second agent is administered in a pyrogen-free, non-oxidized sterile solution having a reducing agent, and optionally a buffer to maintain pH at or near physiologic pH and optionally a metal chelating agent to bind up metal ions that can catalyze oxidation of the thiol-based chemoprotectant agent. Preferably, the thiol-based chemoprotectant agent is stored in a vial having a blanket of an inert gas. Most preferably, the inert gas is selected from the group consisting of argon, helium, nitrogen and mixtures thereof. Preferably, the reducing agent is selected from the group consisting of vitamin E, tocoperol, dithiothreatal, mercaptoethanol, glutathione, and combinations thereof. Preferably, the buffer is one that is relatively non-toxic and can maintain a pH of between 6 and 8 (e.g., phosphate buffer, Tris buffer, Ringers solution, and the like). Preferably, the thiol-based chemoprotectant agent is a compound selected from the group consisting of N-acetyl cysteine (NAC), sodium thiosulfate (STS), GSH ethyl ester, D-methionine, Ethyol, and combinations thereof. Preferably, the daily dose of the thiol-based chemoprotectant agent during chemotherapy is from about 200 mg/m² to about 2000 mg/m². Most preferably, the dose of NAC per procedure is from about 400 mg/m² to about 1200 mg/m².

A pharmaceutical composition for mitigating the gastrointestinal side effects from treatment of carcinoma type cancers with agents that bind to the Lewis Y antigen (administered alone, in combination with other cytotoxic agents, or conjugated to other cytotoxic agents) for administration via arterial catheter is disclosed including an agent administered intra-arterially, wherein the agent is a thiol-based chemoprotectant agent. Preferably, the Lewis Y antigen binding agent is a chimeric monoclonal antibody, optionally conjugated to a cytotoxic agent, and used either alone or in combination with unconjugated, platinum compounds, taxanes (e.g., paclitaxel), steroid derivatives, antimetabolites, vinca alkaloids, adriamycin and doxorubicin, etoposide, arsenic derivatives, intercalating agents, alkylating agents (such as melphalan), and combinations thereof. In a preferred embodiment, the chimeric monoclonal antibody is BR96-Doxorubicin. Preferably, the agent is administered in a pyrogen-free sterile solution Preferably, the agent is administered in a pyrogen-free, non-oxidized sterile solution having a reducing agent, and optionally a buffer to maintain pH at or near physiologic pH and optionally a metal chelating agent to bind up metal ions that can catalyze oxidation of the thiol-based chemoprotectant agent. Preferably, the thiol-based chemoprotectant agent is stored in a vial having a blanket of an inert gas. Most preferably, the inert gas is selected from the group consisting of argon, helium, nitrogen and mixtures thereof. Preferably, the reducing agent is selected from the group consisting of vitamin E, tocoperol, dithiothreatal, mercaptoethanol, glutathione, and combinations thereof. Preferably, the buffer is one that is relatively non-toxic and can maintain a pH of between 6 and 8 (e.g., phosphate buffer, Tris buffer, Ringers solution, and the like). Preferably, the thiol-based chemoprotectant agent is a compound selected from the group consisting of N-acetyl cysteine (NAC), sodium thiosulfate (STS), GSH ethyl ester, D-methionine, Ethyol, and combinations thereof. Preferably, the daily dose of the thiol-based chemoprotectant agent during chemotherapy is from about 200 mg/m² to about 2000 mg/m². Most preferably, the dose of NAC per procedure is from about 400 mg/m² to about 1200 mg/m².

The invention will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF THE DRAWINGS

FIG. 1 shows a graph representing the efficacy of BR96-DOX against human small cell lung carcinoma cells implanted in the brain of the nude rat.

FIG. 2a shows a graph representing the NAC dose response for chemoprotection against the cytotoxicity of alkylating chemotherapeutics.

FIG. 2b shows the dose/response for NAC chemoprotection of BR96-DOX cytotoxicity in normal human gastric cells.

FIG. 3 shows a graph representing protection against BR6-DOX gastric cell toxicity.

FIG. 4 shows a graph representing protection against BR96-DOX gastric cell toxicity when cells are pretreated with BSO.

FIG. 5 shows a graph representing the effect of BSO and NAC on BR96-DOX cytotoxicity.

FIG. 6 shows another graph representing the effect of BSO and NAC on BR-96-DOX cytotoxicity.

FIG. 7 shows a graph representing the effects of BSO on BR96-DOX cyctotoxicity.

FIG. 8 shows another graph representing the effects of BSO on BR96-DOX cyctotoxicity.

FIG. 9 shows an anatomical diagram of major arteries and the top level for placing the catheter for administration of the thiol-based chemoprotectant agent.

### DETAILED DESCRIPTION OF THE INVENTION

Chemoprotection with NAC and/or STS can reduce BR96-DOX toxicity in cultured gastric cells. With its delivery optimized, NAC and/or STS reduce BR96-DOX toxicity in normal GI tract cells in patients, even when the immunoconjugate is given in combination with conventional chemotherapeutic agents.

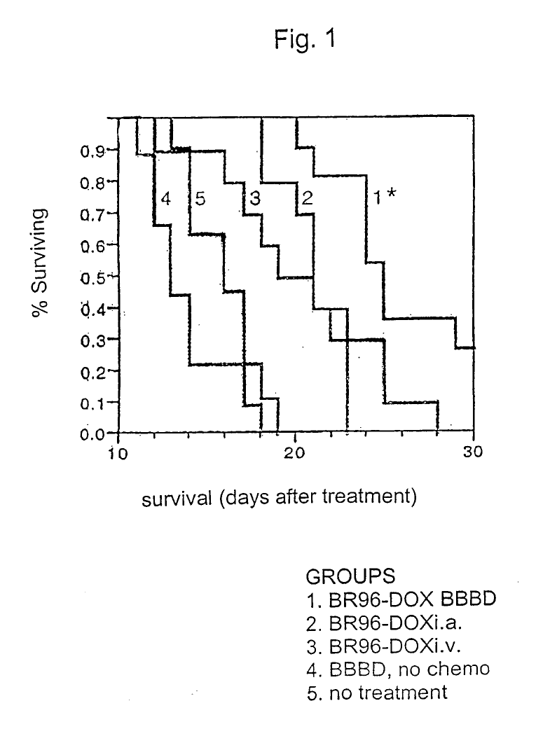

FIG. 1 shows a graph representing the efficacy of BR96-DOX against human small cell lung carcinoma (SCLC) cells implanted in the brain of the nude rat. A Kaplan-Meier survival graph is shown for rats with intracerebral xenografts of B.5 LX-1 cells with low Lewis Y antigen expression. BR96-DOX immunoconjugate was administered with or without optimizing brain delivery using propofol anesthesia. There was a significant increase in survival in animals which received BR96-DOX following osmotic blood brain barrier diffusion (BBBD) (p<0.0001). There was no difference in survival when BR96-DOX was administered either i.a. or i.v. without BBBD, and both groups were significantly better than the controls (BBBD+saline, no treatment, p<0.0001). There was no difference in survival between either control group.

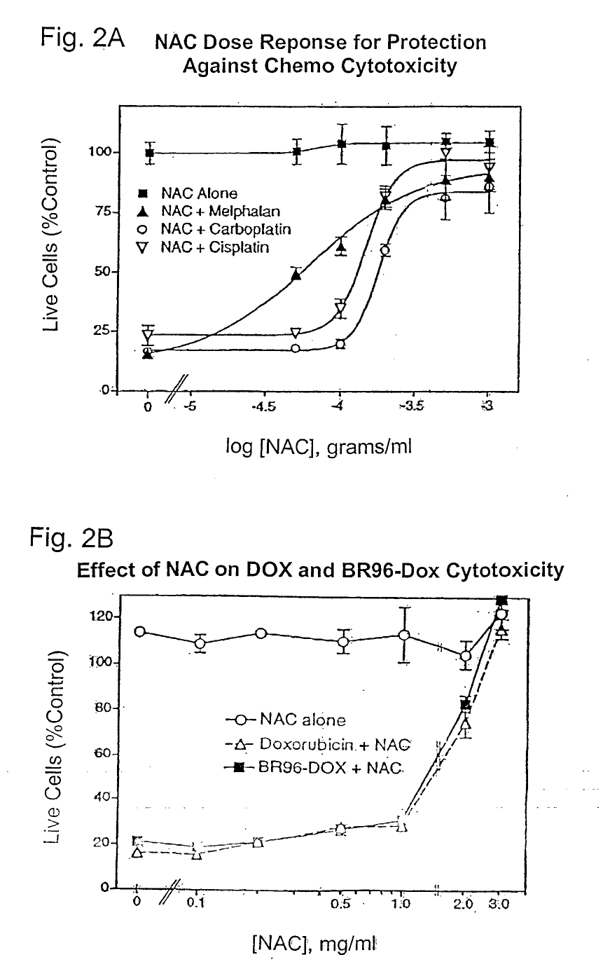

FIG. 2a shows the protection of alkylating chemotherapy cytotoxicity by increasing doses of NAC. Cytotoxicity was assessed in cultured B.5 LX-1 SCLC cells, using the WST colorometric assay for live cells. Cells were treated with approximately LD90 dose of chemotherapy (melphalan=20 μM, carboplatin=200 μM, cisplatin=20 μM). Immediately following chemotherapy, NAC chemoprotectant was added at the indicated concentration. NAC protected against cell death by all three chemotherapeutic drugs, with half-maximal protection found between 0.1 mg/ml NAC and 0.3 mg/ml NAC.

FIG. 2b shows the dose/response for NAC protection against BR96-DOX toxicity in normal human gastric cells. Cytotoxicity was assessed in cultured normal human gastric cells (NHGC), using the WST colorometric assay for live cells. Cells were treated with approximately LD90 dose of BR96-DOX, immediately followed by NAC at the indicated concentrations. NAC was protective against BR96-DOX toxicity in the range of 1 mg/ml to 3 mg/ml, 10-fold higher than the concentration required for chemoprotection against the alkylating chemotherapeuties.

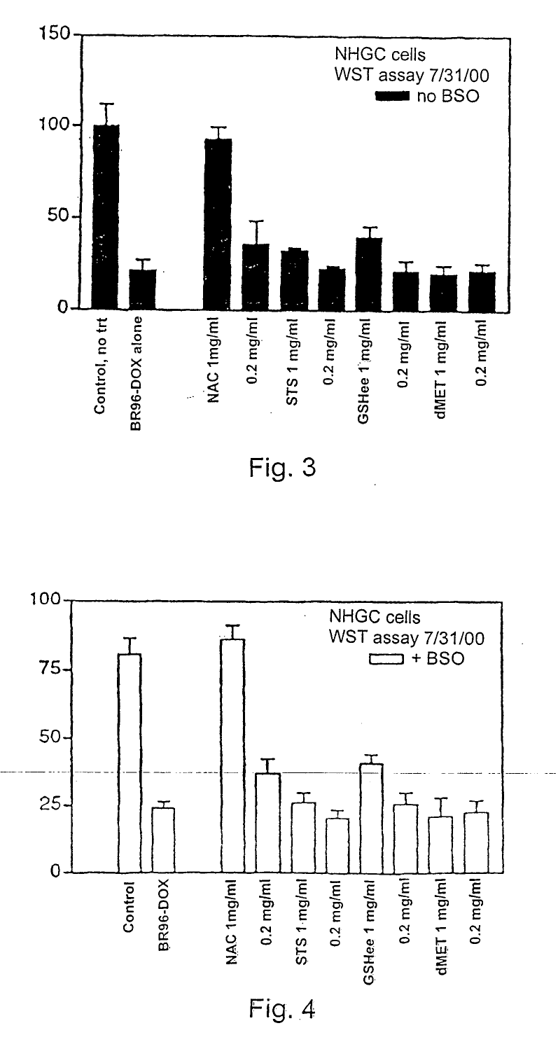

FIGS. 3 and 4 represent graphically the percent viable gastric cells when treated with BR96-DOX alone and in combination with the various alternative chemoprotectants. FIG. 3 records the results without the administration of BSO while FIG. 4 records the results after pretreatment with BSO to reduce intracellular glutathione levels. A greater percentage of viable gastric cells were measured with the administration of NAC and BR96-DOX without the administration of BSO than with the administration of BSO (compare FIG. 3 with FIG. 4, particularly bar number 3 from left). The administration of NAC with BR96-DOX increased the percentage of viable gastric cells regardless of BSO administration. Addition of GSH ethyl ester provided the second highest amount of viable gastric cells.

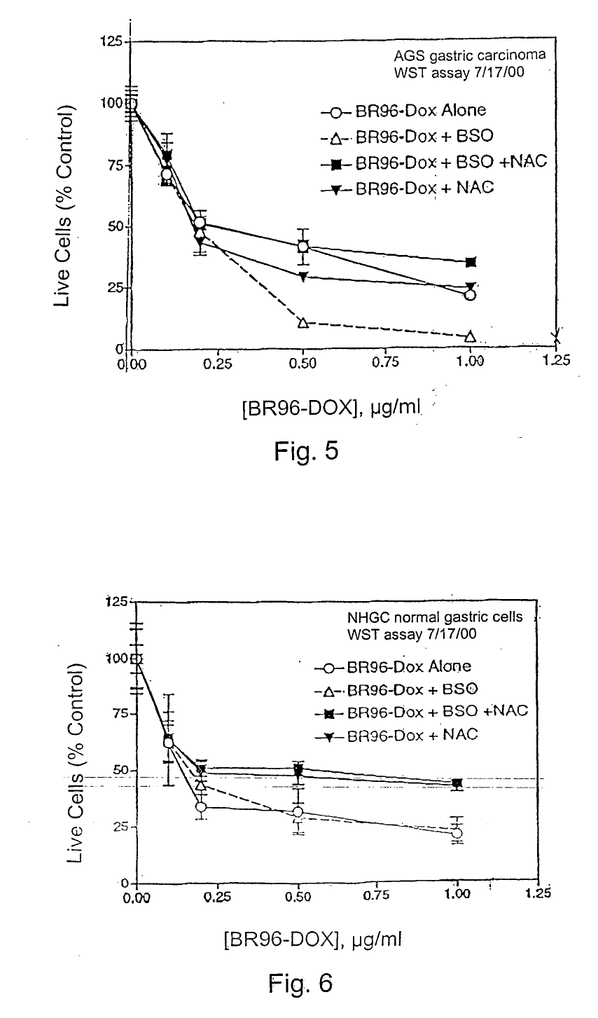

FIGS. 5 and 6 represent the effect of BSO and NAC on BR96-DOX cytotoxicity in gastric carcinoma cells. As the dosage of BR96-DOX is increased, fewer cells survive. The greatest amount of cells survived when the combination of NAC, BSO, and BR96-DOX was administered, compared with the least survival with the administration of BR96-DOX and BSO without NAC.

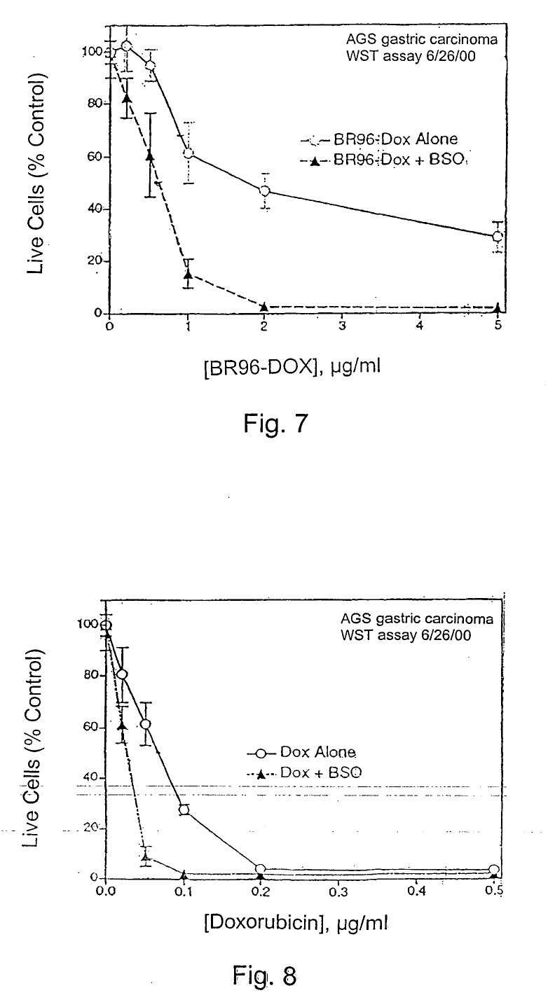

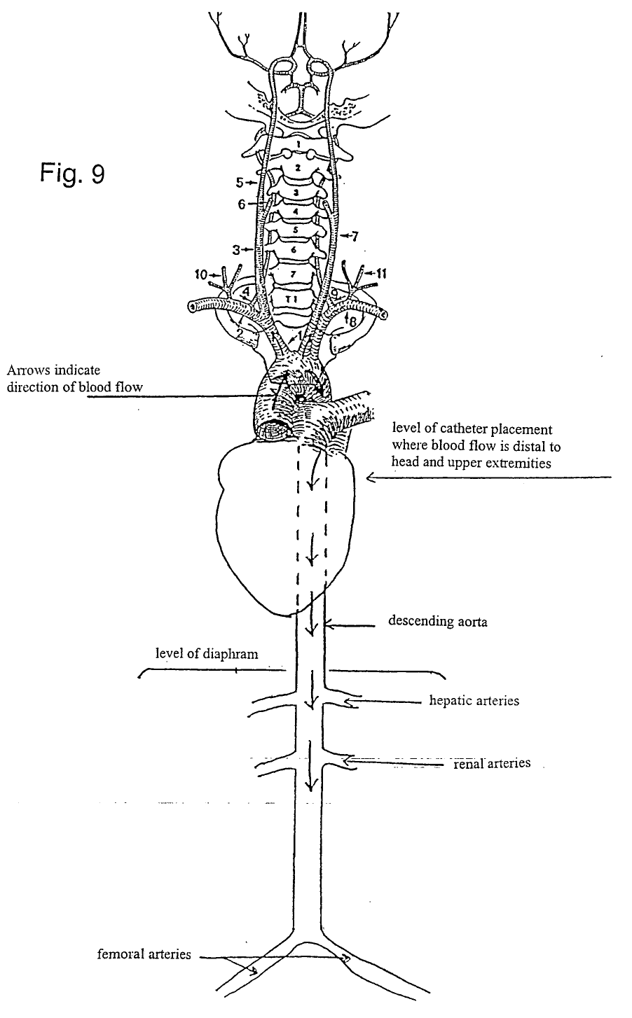

FIGS. 7 and 8 represent the administration of BR96-DOX either alone of in combination with BSO. As expected, the combination of BR96-DOX and BSO reduces the percentage of viable cells to zero.

Modulation of glutathione (GSH) levels may alter the toxicity of chemotherapeutic agents. In vivo cytoenhancement with Buthionine Sulfoximine (BSO) was investigated and found to reduces cellular GSH levels and chemoprotection with N-acetylcysteine (NAC) and sodium thiosulfate (STS); the two later agents can mimic GSH. Modulation of GSH levels with BSO treatment enhances the chemotherapeutic cytotoxicity of intracarotid carboplatin and melphalan. Aortic infusion increases chemoprotectant delivery to systemic tissue with resultant bone marrow protection, but CNS delivery is negligible. In one embodiment, chemoprotection is valuable in the clinical setting if chemotherapy (±Chemo) and chemoprotectant can be physically and/or temporally separated by intra-carotid infusion of alkylators and aortic infusion of chemoprotectant.

Pharmaceutical Formulations

Techniques for the formulation and administration of the compounds of the instant application may be found in “Remington's Pharmaceutical Sciences” Mack Publishing Co., Easton, Pa., latest addition. Suitable routes of administration are intra-arterial.

The compositions and compounds of the present invention may be manufactured in a manner that is itself known, e.g., by means of conventional mixing, dissolving, emulsifying, encapsulating, entrapping, or lyophilizing processes. Pharmaceutical compositions for use in accordance with the present invention thus may be formulated in conventional manner using one or more physiologically acceptable carriers comprising excipients and auxiliaries that facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers, such as Hank's solution, Ringer's solution, or physiological saline buffer. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulary agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

A therapeutically effective dose refers to that amount of the compound that results in a reduction in the development or severity of reduction in renal function. Toxicity and therapeutic efficacy of such compounds can be determined by standard pharmaceutical, pharmacological, and toxicological procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from cell culture assays or animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. The individual physician in view of the patient's condition can choose route of administration and dosage the exact formulation. (Fingl et al., 1975, in “The Phramacological Basis of Therapeutics”, Ch. 1).

The amount of composition administered will, of course, be dependent on the subject being treated, on the subject's weight, the severity of the affliction, the manner of administration and the judgment of the prescribing physician.

The thiol-based chemoprotectant agent is administered intra-arterially according to the present invention and in order for systemic tissues to be exposed to an initial dose of the thiol-based chemoprotectant agent in high enough concentration by chemoprotective effective effect and before getting to the venous circulation and being eliminated by the liver.

Synthesis

Each thiol-based chemoprotectant agent, such as NAC or STS, can be synthesized by conventional methods and are commercially available as a sterile solution. Pyrogen-free solutions for intra-arterial administration and those with buffers for physiologic pH administrations can be made by conventional techniques.

The results of the following examples suggest that cyto-enhancement and chemoprotection may be effective in combination with BR96-DOX treatment. A NHGC cell line and a human gastric carcinoma cell line (AGS) were obtained. Both cell lines were homogeneously highly positive for immunocytochemical staining with the BR96 antibody directed against the Lewis Y antigen. BSO enhanced cytotoxicity in the carcinoma cells, but did not increase toxicity in the normal gastric cells, the site of dose-limiting toxicity of BR96-DOX. Conversely, NAC protected the normal gastric cells from BR96-DOX toxicity, but did not protect the carcinoma cells.

### EXAMPLE 1

The dose response curves for BR96-DOX and doxorubicin with or without the addition of buthionine sulfoximine (BSO) at a concentration of 100 μM were assessed. The half maximal cytotoxic dose of BR96-DOX administered to AGS cells was approximately 1 μg/ml in the absence of BSO. Pretreatment with BSO reduced the EC50 to approximately 0.6 μg/ml. BSO treatment also increased the maximum cytotoxicity of BR96-DOX from 70% to 100% cell kill. Pretreatment with BSO also shifted the half maximal cytotoxic dose of doxorubicin from 0.1 μg/ml to approximately 0.05 μg/ml, but did not enhance the maximum cytotoxicity of doxorubicin, as doxorubicin alone killed nearly 100% of cells at maximal doses. In a second experiment, BSO did not significantly shift the EC50 of BR96-DOX in AGS carcinoma cells grown in an unsupplemented medium, but did increase the maximal cell kill from 75% to 100%.

In NHGC cells, as well as in the low-expressor and high-expressor subclones of the LX-1 SCLC cell line, BSO did not enhance the cytotoxicity of either BR96-DOX or doxorubicin.

### EXAMPLE 2

Chemoprotection in the gastric cells was examined by using NHGC normal gastric cells wherein the chemoprotective agent N-acetylcysteine (NAC) was at least partially protective against BR96-DOX cytotoxicity. The level of protection was variable, reducing cell kill by 25% in experiment 1, 95% in experiment 2, and 55% in experiment 3. NAC was protective independent of the presence of BSO. Other chemoprotective agents tested were not as effective as NAC, with only GSH ethyl ester yielding significant protection (cell kill reduced by 15-20%) and no significant effect of sodium thiosulfate or d-Methionine.

In contrast to the NHGC cells, NAC was not signficantly effective at reducing BR96-DOX cytotoxicity in the AGS gastric carcinoma cells. NAC did reverse the enhanced cytotoxicity induced by BSO treatment, but did not alter the response to BR96-DOX in cells not treated with BSO.

### EXAMPLE 3

NAC biodistribution was determined with radiolabelled tracer (n=12). Blood and tissue GSH levels were measured with a colorometric kit 9 (n=19). For bone marrow toxicity studies, rats were treated with or without BSO (10 g/m² i.p. b.i.d.×3 days), followed by chemotherapy consisting of intra-carotid carboplatin (200 mg/m²), melphalan (10 mg/m²) and etoposide phosphate (100 mg/m²) (n=61). The dose of NAC was 1200 mg/m² and STS was 8 gm/m². White blood cell and platelet counts were obtained prior to, at 6 days and 9-10 days after chemotherapy. BSO treatment for 3 days reduced blood and tissue GSH levels by 50-65% even in brain and intracerebral tumor in nude rats. BSO pretreatment enhanced the bone marrow toxicity of combination chemotherapy. Intraarterial administration of radiolabelled NAC in the right carotid artery resulted in high delivery to the right cerebral hemisphere, however, infusion of NAC via a new “aortic infusion” technique, retrograde in the left external carotid artery with the left internal carotid artery occluded to prevent infusion of the brain, reduced brain delivery to negligible levels while increasing systemic delivery. When NAC was administered via “aortic infusion” before intra-carotid chemotherapy (no BSO), the magnitude of the bone marrow toxicity nadir at day 6 was markedly reduced (no NAC: platelets 215±126, granulocytes 146±160; with NAC: platelets 470±234, granulocytes 785±494, which by non-parametric analysis gave a p value of <0.02). Virtually no myelosuppression occurred if both NAC and STS were given via “aortic infusion” even in BSO-treated animals.

The discussion above is descriptive, illustrative and exemplary and is not to be taken as limiting the scope defined by any appended claims.

I claim:

1. A method for treating or mitigating the side effects of a cytotoxic cancer therapy for carcinoma type cancers comprising:
administering at least a cytotoxic agent and a thiol-based chemoprotectant agent intra-arterially, wherein the intra-arterial administration is through a catheter placed into an artery that provides blood flow to an organ most susceptible to toxic side effects of the cytotoxic agent.

2. The method of claim 1 wherein the thiol-based chemoprotectant agent is selected from the group consisting of N-acetyl cysteine (NAC), sodium thiosulfate (STS), GSH ethyl ester, D-methionine, ethyol, and combinations thereof.

3. The method of claim 1 wherein the cytotoxic agent is selected from the group consisting of chimeric anti-Lewis Y monoclonal antibodies conjugated to a cytotoxic agent, alone or in combination with unconjugated platinum compounds, taxanes, steroid derivatives, anti-metabolites, vinca alkaloids, adriamycin and doxorubicin, etoposide, arsenic derivatives, intercalating agents, alkylating agents and combinations thereof.

4. The method of claim 1 wherein the dose of the thiol-based chemoprotectant agent per procedure is from about 200 mg/m² to about 40 g/m².

5. The method of claim 1 wherein the cytotoxic agent is a monoclonal antibody to the Lewis Y glycoprotein.

6. The method of claim 5 wherein the monoclonal antibody is the BR96-doxorubicin immunoconjugate.

7. The method of claim 4 wherein the daily dose of NAC agent during chemotherapy is from about 400 mg/m² to about 1200 mg/m².

8. A pharmaceutical composition for treatment of carcinoma type cancers for administration via arterial catheter comprising:
a first agent that is a cancer cytotoxic agent, and a second agent administered intra-arterially, wherein the first agent is a cytotoxic compound that is used for cancer chemotherapy but is dose-limited due to side effects, and the second agent is a thiol-based chemoprotectant agent.

9. The pharmaceutical composition of claim 8 wherein the first agent is selected from the group consisting of chimeric anti-Lewis Y monoclonal antibodies conjugated to a cytotoxic agent, alone or in combination with unconjugated platinum compounds, taxanes, steroid derivatives, anti-metabolites, vinca alkaloids, adriamycin and doxorubicin, etoposide, arsenic derivatives, intercalating agents, alkylating agents, and combinations thereof.

10. The pharmaceutical composition of claim 9 wherein the chimeric monoclonal antibody is BR96-Doxorubicin.

11. The pharmaceutical composition of claim 8 wherein the second agent is administered in a pyrogen-free sterile solution.

12. The pharmaceutical composition of claim 8 wherein the thiol-based chemoprotectant agent is selected from the group consisting of N-acetyl cysteine (NAC), sodium thiosulfate (STS), GSH ethyl ester, D-methionine, ethyol, and combinations thereof.

13. The pharmaceutical composition of claim 8 wherein the daily dose of the thiol-based chemoprotectant agent during chemotherapy is from about 200 mg/m² to about 2000 mg/m².

14. The pharmaceutical composition of claim 12 wherein the thiol based chemoprotectant agent is NAC.

15. The pharmaceutical composition of claim 13 wherein the dose of NAC per procedure is from about 400 mg/m² to about 1200 mg/h².

16. A pharmaceutical composition for mitigating the gastrointestinal side effects from treatment of carcinoma type cancers with agents that bind to the Lewis Y antigen, administered alone, in combination with other cytotoxic agents, or conjugated to other cytotoxic agents, for administration via arterial catheter comprising:
an agent administered intra-arterially, wherein the agent is a thiol-based chemoprotectant agent.

17. The pharmaceutical composition for mitigating the gastrointestinal side effects from treatment of carcinoma type cancers of claim 16 wherein the Lewis Y antigen binding agent is a chimeric monoclonal antibody.

18. The pharmaceutical composition for mitigating the gastrointestinal side effects from treatment of carcinoma type cancers of claim 16 wherein the Lewis Y antigen binding agent is conjugated to a cytotoxic agent.

19. The pharmaceutical composition for mitigating the gastrointestinal side effects from treatment of carcinoma type cancers of claims 15, 16 or 17 wherein the Lewis Y antigen binding agent is used either alone or in combination with unconjugated, platinum compounds, taxanes, steroid derivatives, anti-metabolites, vinca alkaloids, adriamycin and doxorubicin, etoposide, arsenic derivatives, intercalating agents, alkylating agents, and combinations thereof.

20. The pharmaceutical composition for mitigating the gastrointestinal side effects from treatment of carcinoma type cancers of claim 17 wherein the chimeric monoclonal antibody is BR96-Doxorubicin.

21. The pharmaceutical composition for mitigating the gastrointestinal side effects from treatment of carcinoma type cancers of claim 16 wherein the agent is administered in a pyrogen-free sterile solution.

22. The pharmaceutical composition for mitigating the gastrointestinal side effects from treatment of carcinoma type cancers of claim 21 further including a buffer capable of maintaining pH at or near physiologic pH.

23. The pharmaceutical composition for mitigating the gastrointestinal side effects from treatment of carcinoma type cancers of claim 16 further including a metal chelating agent capable of binding metal ions that can catalyze oxidation of the thiol-based chemoprotectant agent.

24. The pharmaceutical composition for mitigating the gastrointestinal side effects from treatment of carcinoma type cancers of claim 16 wherein the thiol-based chemoprotectant agent is stored in a vial having a blanket of an inert gas.

25. The pharmaceutical composition for mitigating the gastrointestinal side effects from treatment of carcinoma type cancers of claim 24 wherein the inert gas is selected from the group consisting of argon, helium, nitrogen and mixtures thereof.

26. The pharmaceutical composition for mitigating the gastrointestinal side effects from treatment of carcinoma type cancers of claim 16 further including a reducing agent.

27. The pharmaceutical composition for mitigating the gastrointestinal side effects from treatment of carcinoma type cancers of claim 26 wherein the reducing agent is selected from the group consisting of vitamin E, tocoperol, dithiothreatal, mercaptoethanol, glutathione, and combinations thereof.

28. The pharmaceutical composition for mitigating the gastrointestinal side effects from treatment of carcinoma type cancers of claim 22 wherein the buffer is relatively non-toxic and can maintain a pH of between 6 and 8.

29. The pharmaceutical composition for mitigating the gastrointestinal side effects from treatment of carcinoma type cancers of claim 28 wherein the buffer is selected from the group consisting of phosphate buffer, Tris buffer, Ringers solution, and combinations thereof).

30. The pharmaceutical composition for mitigating the gastrointestinal side effects from treatment of carcinoma type cancers of claim 16 wherein the thiol-based chemoprotectant agent is a compound selected from the group consisting of N-acetyl cysteine (NAC), sodium thiosulfate (STS), GSH ethyl ester, D-methionine, Ethyol, and combinations thereof.

31. The pharmaceutical composition for mitigating the gastrointestinal side effects from treatment of carcinoma type cancers of claim 16 wherein the daily dose of the thiol-based chemoprotectant agent during chemotherapy is from about 200 mg/m² to about 2000 mg/m².

32. The pharmaceutical composition for mitigating the gastrointestinal side effects from treatment of carcinoma type cancers of claim 30 wherein thiol-based chemoprotectant agent is NAC.

33. The pharmaceutical composition for mitigating the gastrointestinal side effects from treatment of carcinoma type cancers of claim 32 wherein the dose of NAC per procedure is from about 400 mg/m² to about 1200 mg/m².

34. The pharmaceutical composition formulating the gastrointestinal side effects from treatment of carcinoma type cancers of claim 19 wherein the alkylating agent is selected from the group consisting of melphalan, carboplatin, cisplatin and combinations thereof.
